(19) United States
(12) Patent Application Publication
(10) Pub. No.: US 2014/0296190 A1
(43) Pub. Date: Oct. 2, 2014

(54) MARKETING THE USE OF 25-HYDROXYVITAMIN D3 TO ENHANCE COGNITION

(71) Applicant: DSM IP ASSETS B.V., (US)

(72) Inventors: Elske Maria BROUWER-BROLSMA, Wageningen (NL); Edith Johanna Maria Feskens, Utrecht (NL); Lisette CPGM De Groot, Poppel (BE); Stephanie J.M. Krammer-Lukas, Basel (CH); Hasan Mohajeri, Basel (CH); Szabolcs Peter, Basel (CH); Jonas Wittwer Schegg, Basel (CH)

(73) Assignee: DSM IP ASSETS B.V., Basel (CH)

(21) Appl. No.: 13/851,463

(22) Filed: Mar. 27, 2013

(51) Int. Cl. A61K 31/593

(52) U.S. Cl. CPC A61K 31/593
USPC 514/167

(57) ABSTRACT

This invention relates to a method of marketing 25-hydroxyvitamin D3 (25-OH D3) comprising providing an effective dose of 25-OH D3 and informing the potential consumer that it is beneficial for increasing or maintaining executive functioning, or lessening the decrease of cognitive functioning. Kits containing the 25-OH D3 and information regarding its benefits in this area are also part of this invention.

### BRIEF DESCRIPTION OF THE INVENTION

This invention relates to a business method, mainly to the method of marketing 25-hydroxyvitamin D (“25-OH D3”), either alone or in combination with Vitamin D3 for the purpose of enhancing and/or maintaining certain aspects of cognition in humans, particularly in the area of executive functioning.

### BACKGROUND OF THE INVENTION

Aging has been shown to be one of the risk factors for the development of vitamin D deficiency. It is also generally known that ageing is a key risk factor for cognitive decline and dementia. Mice lacking vitamin D receptors (VDRs) demonstrate an increased anxiety level, inferior nest building, and impaired motor performance, which suggests a role for vitamin D in various brain processes. Mechanistically, there appears to be an effect of vitamin D on intraneuronal calcium regulation and the synthesis and degradation of several neurotransmitters and neurotrophins. Further, Vitamin D has been suggested to beneficially affect amyloid p phagocytosis and clearance by macrophages.

Although a recent review by Eyles et al. 2012 Front. Neuroendocrinol. (http://dx.doi.org/10.1016/j.yfrne.2012.07.001) showed that a role for vitamin D in certain brain function is confirmed by neurobiological evidence, they also concluded that there is still insufficient and inconsistent data from epidemiological studies and well-designed randomized controlled trials in humans to verify this.

Thus, already several human population based studies investigated the possible role of 25(OH)D in cognitive performance, although none of these describe intervention trials:

Annweiler et al 2009 Eur J Neurol 16(10):1083-1089 looked at the literature for studies showing a significant positive correlation between 25-OH D3 levels and cognitive performance. The results were inconsistent.

Breitling et al 2012 Experimental Gerontology 47(1):122-127 found that low vitamin D levels are associated with worse cognitive function in the elderly assessed after 5 years. High levels of vitamin D showed a plateau of cognitive performance functioning.

Brouwer-Brolsma et al 23 Jun. 3 2012 Eur J. Nutr. online publication DOI 10.1007/s00394-012-0399-0 found inverse associations between 25-OH D3 plasma levels and fasting glucose levels, and there was a tendency for lower depression levels to be associated with higher 25-OH D3 serum levels. Data did not support the hypothesis that higher 25-OH D3 levels were associated with better cognitive functioning. There was no information reported specifically on executive functioning.

Chan et al. J Affect Disord. April 2011; 130(1-2):251-259. Serum levels of 25-OH D3 were found to be inversely associated with depression. However, no association was found between 25-OH D3 levels and cognitive impairment. Specific effects on executive functioning were not reported.

Seamans et al 2010 Eur. J. Clin Nutr. 4(10):1172-1178 looked at associations between serum 25-OH D3 status and various aspects of cognitive function. There was an association between 25-OH D3 levels in some aspects of spatial working memory, but not all such aspects.

Lee et al 2009. J Neurol Neurosurg Psychiatry 80(7):722-729 found that lower scores on the Digital Symbol Substitution Test were associated with lower 250H D3 levels, but this correlation was not seen for other cognitive tests.

WO 1995/02409 (Trustees of the Univ of Kentucky) disclose use of vitamin D, its metabolites and precursors to protect against neuron loss, such as is observed in Alzheimers and other neuronal based diseases.

It would be desirable to provide a safe, effective way to enhance or retain cognitive executive functioning in healthy people.

### DETAILED DESCRIPTION OF THE INVENTION

It has been found, in accordance with this invention that serum 25-hydroxyvitamin D3 (25-OH D3) levels are positively associated with maintaining or enhancing the cognitive processes referred to as “executive function”, or lessening the decrease of executive function in healthy individuals. However, it has also been found, in accordance with this invention, that there is not always a significant correlation between vitamin D intake and serum 25-OH D3 levels, particularly in elderly people; and it is presumed sunlight exposure may be more influential.

Thus one aspect of this invention is the method of marketing 25-OH D3 to maintain or enhance executive functioning in healthy individuals or lessen the risk of a decline in executive functioning in healthy individuals.

Another aspect of this invention is directed to a method of marketing 25-OH D3 for the enhancing, maintaining of executive functioning in healthy individuals or for lessening the decrease of cognitive executive function comprising:
providing to a healthy human desirous of enhancing or maintaining cognitive-executive-function, or desirous of lessening the decrease of cognitive function an effective amount of 25-OH D3; and
and providing information concerning the benefits of 25-OH D3 for the purpose of enhancing or maintaining cognitive-executive-function, or desirous of lessening the decrease of cognitive function.

Another aspect of this invention is directed to a method of marketing 25-OH D3 of enhancing, or maintaining cognitive executive function performance comprising:
providing a healthy human desirous of enhancing or maintaining cognitive-executive-function an effective amount of 25-hydroxyvitamin D3 and informing the individual about the ability of 25-OH D3 to enhance, or maintain executive function prior to an event that would challenge the healthy human's executive functioning ability; and
challenging the healthy human with an event requiring executive functioning, and
assessing the individual's resulting enhanced or maintained executive function.

Another aspect of this invention is a method of lessening the decrease of executive functioning in a healthy individual comprising providing an effective amount of 25-OH D3 to the individual desirous of lessening the decrease of executive functioning, prior to an event that would challenge the healthy human's executive functioning ability; and
challenging the healthy human with an event requiring executive functioning, and
assessing the individual's resulting lessened decrease of executive function.

This invention also relates to a method of marketing 25-OH D3 for maintaining or enhancing executive functioning, or lessening the decrease executive function comprising providing a kit comprising:
a) at least one oral dosage form of 25-OH D3; and
b) information which informs a prospective user about the benefits of using 25-OH D3 for enhancing or maintaining cognitive executive function in healthy individuals, or information which informs a prospective user about the benefits of using 25-OH D3 to lessen the decrease of executive function.

The enhancing or maintaining of cognitive executive function, or the lessening the decrease of cognitive function by use of orally administered 25-OH D3 may optionally further comprise the use of Vitamin D in combination with the 25-OH D3.

This invention also relates to a method of marketing 25-OH D3 supplemented food for maintaining or enhancing executive function, or lessening the decrease executive function comprising providing a kit comprising:
a) food which has comprises an effective amount of 25-OH D3; and
b) information which informs a prospective user about the benefits of using 25-OH D3 in enhancing or maintaining cognitive executive function in healthy individuals, or which informs a prospective uses about the benefits of using 25-OH D3 to lessen the decrease of executive function.

### DEFINITIONS

As used throughout the specification and claims, the following definitions apply:

Executive function—this is an umbrella term for cognitive processes that regulate, control and manage other cognitive processes, including planning, working memory, attention, problem solving, verbal reasoning, inhibition, mental flexibility, task switching, and initiation and monitoring of actions. The prefrontal areas of the frontal lobe are necessary but not sufficient for carrying out these functions.

Executive system—is a theorized cognitive system in psychology that controls and manages other cognitive processes. It is responsible for processes that are sometimes referred to as executive functions, executive skills, supervisory attentional system, or cognitive control. The prefrontal areas of the frontal lobe are necessary but not sufficient for carrying out these functions.

Healthy individual—when used in context of this invention, the healthy individual does not have any of the following conditions: depression, Alzheimer's Disease, schizophrenia, autism, Parkinson's disease, psychotic conditions (such as sub clinical schizophrenia), Alzheimer's-like dementia, AIDS-dementia complex, other dementias, glucose intolerance, diabetes, or conditions requiring kidney or liver transplants. Further, a “healthy individual” has not previously received 25-OH D3 for the treatment of osteoporosis, or physical conditions related to Vitamin D deficiency.

“Vitamin D” means either Vitamin D3 (cholecalciferol) and/or Vitamin D2 (ergocalciferol). Humans are unable to make Vitamin D2 (ergocalciferol), but are able to use it as a source of Vitamin D. Vitamin D2 can be synthesized by various plants and is often used in Vitamin D in supplements as an equivalent to Vitamin D3

“Prevent” is meant to include amelioration of the disease, lessening of the severity of the symptoms, early intervention, and lengthening the duration of time prior to the onset of the disease, and is not intended to be limited to a situation where the patient is unable to experience any symptoms of executive function impairment.

“Vitamin D deficient” means that the serum levels of 25-OH D3 are between 25 to 49 nmol/l. Amounts less than 25 nmol/l are considered severely deficient.

“Vitamin D sufficient” means that the serum levels of 25-OH D3 are between 50 to 75 nmol/l; levels above 75 nmol/l are generally considered desired or optimal.

The kit which is provided may be comprised of multiple, separate dosages of 25-OH D3 and optionally, Vitamin D3. The separate dosages may be enclosed in a container: e.g., bottle, blister pack, or vial rack. Further, instructions for administering the composition as a dosage to a human are preferably also provided as part of the kit.

In one embodiment, the kit comprises dosage forms such as a plurality of capsules, tablets or sachets which may comprise:
a) 25-OH D3 as the sole active ingredient,
b) 25-OH D3 and vitamin D as sole active ingredients. The 25-OH D3 may be admixed with the vitamin D, or the two active ingredients may be separate, but both are administered at essentially the same time. “Essentially the same time” is intended to mean that the two active ingredients are taken simultaneously, serially, or within 10 minutes of each other.
c) 25-OH D3 or the combination of 25-OH D3 plus vitamin D may also be administered in a formulation with a further active ingredient(s). The further active ingredients may include compounds known to enhance an aspect of cognitive function or may provide different benefits.

The capsules, tablets or sachets or other dosage forms may be in a container which may take any conventional form. For example the dosage forms may be sold in a jar, bottle, tin box, pot, or the like which contains the dosage forms in a predetermined quantity, such as a 30-day supply, a 60-day supply, a 90-day supply or in whatever quantity which is desired. Additionally and optionally, the capsules may be in a blister pack, wherein each blister contains a predetermined number of capsules, usually a single dose (typically 1-4 capsules). The arrangement of the number of capsules in a blister, the number of blisters on a single blister pack strip, and the number of blister pack strips which are sold in a group may be any convenient amounts or configurations.

Informational material may be part of the material used to package the dosage forms. For example, if the dosage forms are packaged in a container which may be re-opened and closed, such as a jar, bottle, or pot, then the informational material may be printed on a label which is affixed to the outside of the container. Alternatively and/or additionally, the informational material may be on a separate insert which is placed in a box, envelope, or the like which holds the container. In instances where the dosage forms are packaged in a blister pack or the like, the informational material may be printed on the blister pack strip, on a receptacle such as a box or envelope containing the blister pack strips, and/or included in a package insert placed inside the box or envelope containing the blister packs. The exact form of the informational material is not critical to this invention as long as the information provided informs the user as to the benefits of 25-OH D3 to maintain, enhance or lessen the decrease of at least one aspect of executive functioning.

Alternatively and/or additionally, the informational material need not be physically associated with the kit. For example, the informational material may be in the form of printed leaflets, flyers, advertising placards, or the like which is displayed in the proximity (preferably within one meter) of the kit. The informational material may be in a form which allows the potential consumer to take a printed material (such as a hand-out, flier, postcard or the like) or may merely impart information. The information may be provided to the customer through conventional marketing methods using a variety of media, such as though mass communication advertising (television/radio advertising, print advertising such as in magazines, internet marketing and advertising such as through web sites, social networking sites, and the like). The important point is that the consumer is informed about at least one benefit of the use of 25-OH D3 either alone or in combination with vitamin D3 to enhance, or maintain at least one aspect of executive functioning, or to lessen a decrease in at least one aspect of executive functioning.

The peak concentration of 25-OH D achieved by such administration may be from 30 nmol/L to 375 nmol/L, preferably from about 120 nmol/L to about 300 nmol/L. The steady-state concentration of 25-OH D achieved by such administration is preferably from above 60 nmol/L.

### Information Imparted

The information which is to be imparted, should include at least one of the following benefits. The exact wording to the benefit is not critical to this invention, as long as the prospective purchaser is informed of the benefit, but should impart information that 25-OH D3 can enhance, or maintain or, alternately, lessen the decrease of at least one executive function. This may include at least one of the following attributed of executive functioning:
Planning: foresight in devising multi-step strategies.
Flexibility: capacity for quickly switching to the appropriate mental mode.
Inhibition: the ability to withstand distraction, and internal urges.
Anticipation: prediction based on pattern recognition.
Critical evaluation: logical analysis.
Working memory: capacity to hold and manipulate information “on-line” in our minds in real time.
Fuzzy logic: capacity to choose with incomplete information.
Divided attention: ability to pay attention to more than one thing at a time.
Decision-making: both quality and speed

Additionally, the information may also include items such as dosages, instructions on how to consume the dosages, ingredients (both active and inert) of the dosages, and expiry date.

People who would potentially benefit from enhancing executive function or maintaining healthy executive function would include the following categories, and the information may be specifically tailored to consumers of the following groups:

a) People with learning disabilities (LD) and attention-deficit hyperactivity disorder—(ADHD). Both LD and ADHD have been associated with executive dysfunction. Therefore otherwise healthy people who experiencing ADHD or LD can benefit from oral administration of 25-OH D3.
b) People employing academic skills—executive function is involved in academic skills such as reading comprehension, writing essays or writing projects, and taking tests. Increasing executive function according to this invention would be useful for students, people learning new subjects, people who need to organize information for their jobs, people who write reports, and people who need to take examinations, and the like.
c) People involved with long term projects—executive function is involved in long term planning and completing complex tasks. For example, the ability to time manage, set realistic milestones, monitoring progress during a project, and organizing projects are all linked to executive function. Thus, people who are involved in managing projects or working on long-term or complicated or multistep activities would benefit from this invention.
d) People who shifting between tasks—the ability to “multitask”, i.e. being able to shift rapidly between two or more tasks without becoming confused is also a type of executive function. Thus people who are expected to multitask could benefit from 25-OH D3.

Thus, this invention would also be of particular interest to the following healthy groups of people who are desirous in preserving, enhancing, or maintaining their executive functioning, including, but not limited to: aging people, students, teachers, researchers, academics, writers, editors, lawyers, politicians, artists, performing artists (such as in theatre, radio, opera, cinema or circus), choreographers, directors, those facing a test or examination, those who organize events, tasks, and or processes, such as project planners, office managers, people working in the service industries (such as restaurant personnel, hoteliers, and the tourist industries) military commanders, other type of coordinators, people expected to carry out multitasking, such as air traffic controllers, pilots, teachers, service personnel, builders, architects, medical personnel, child care providers, and/or managers.

Another aspect of this invention is that orally administered 25-OH D3, either alone or in combination with Vitamin D3 can increase serum plasma levels, and thus maintain, enhance and/or prevent the lessening of executive function. The combination of both active ingredients may be particularly advantageous as it 1) It results in a rapid and synergistic plasma response of 25-OH D; and 2) it leads to an pronounced and long plateau of plasma 25-OH D levels.

### Formulations

The nutraceutical and pharmaceutical compositions according to the present invention may be in any galenic form that is suitable for administering to humans, but oral forms are preferred, e.g. in solid form, such as additives/supplements for food, food, fortified food or feed, tablets, pills, granules, dragées, capsules, gummy formulations, and effervescent formulations such as powders and tablets, or in liquid form such as solutions, emulsions or suspensions as e.g. beverages, pastes and oily suspensions. The pastes may be encapsulated in hard or soft shell capsules, whereby the capsules feature e.g. a matrix of (fish, swine, poultry, cow) gelatin, plant proteins or lignin sulfonate. The dietary and pharmaceutical compositions may be in the form of controlled (delayed) release formulations.

The dietary compositions according to the present invention may further contain protective hydrocolloids (such as gums, proteins, modified starches), binders, film forming agents, encapsulating agents/materials, wall/shell materials, matrix compounds, coatings, emulsifiers, surface active agents, solubilizing agents (oils, fats, waxes, lecithins etc.), adsorbents, carriers, fillers, co-compounds, dispersing agents, wetting agents, processing aids (solvents), flowing agents, taste masking agents, weighting agents, gelling agents, gel forming agents, antioxidants and antimicrobials.

In addition the pharmaceutical or nutraceutical compositions according to the present invention may further contain conventional pharmaceutical additives and adjuvants, excipients or diluents, including, but not limited to, water, gelatin of any origin, vegetable gums, ligninsulfonate, talc, sugars, starch, gum arabic, vegetable oils, polyalkylene glycols, flavoring agents, preservatives, stabilizers, emulsifying agents, buffers, lubricants, colorants, wetting agents, fillers, and the like. The carrier material can be organic or inorganic inert carrier material suitable for oral/parenteral/injectable administration.

Examples of food which may be supplemented or fortified with 25-OH D3 or optionally both 25-OH D3 and vitamin D include cereal bars, dairy products, such as yoghurts, and bakery items, such as breads, cakes and cookies, non-alcoholic drinks, such as soft drinks, fruit juices, lemonades, near-water drinks, teas and milk-based drinks, in the form of liquid food, such as soups and dairy products (muesli drinks).

### Dosages

The combination of vitamin D and 25-OH D3 may be administered once per day, once per week, or once per month. Delivering a combination of the vitamin D and 25-OH D3, results in plasma levels of 25-OH D3 increase synergistically. This effect is observed rapidly, and is most pronounced after about the first 6 hours. Further, the increase in plasma levels is sustained (albeit at a lower, but still clinically effective level), for at least approximately 206 hours. The rapid effect provides acute bioavailability, while the sustained elevated plasma levels ensures extended bioavailability.

Daily. A composition according to this invention where the two active ingredients are to be administered in separate dosage forms, contains Vitamin D or 25-OH D3 in an amount from about 1 μg to about 50 μg, preferably about 5 μg and 25 μg. Alternatively, a single daily dosage having both Vitamin D and 25-OH D3 contains each active ingredient in an amount from about 1 μg to about 50 μg, preferably about 5 μg and 25 μg. Where 25-OH D3 is the only active ingredient with vitamin D-type activity, then its dosage is about 1 μg to about 50 μg.

The dosage ratio of Vitamin D to 25-OH D3 may be from about 50:1 to about 1:50, more preferably from about 25:1 to about 1:25, and even more preferably from about 6:1 to about 1:6.

Multiple, separate dosages may be packaged in a single kit (or container). For example, the kit may be comprised of thirty separate daily dosages of both actives separately (i.e. 60 separate dosages), or combined (i.e. 30 dosages containing both active ingredients). Instructions for administering the dosages to a human may be included in the kit.

Weekly. A single weekly dosage contains 25-OH D3 in an amount from about 7 μg to about 350 μg, and preferably from about 35 to 175 μg. Alternatively, a single weekly dosage may contain both Vitamin D and 25-OH D3 each in an amount from about 7 μg to about 350 μg, and preferably from about 35 to 175 μg. The dosage ratio of Vitamin D to 25-OH D3 may be from about 50:1 to about 1:50, more preferably from about 25:1 to about 1:25, and even more preferably from about 6:1 to about 1:6.

Monthly. A single monthly dosage contains Vitamin D or 25-OH D3 in an amount from 30 μg to about 1500 μg, preferably about 75 μg to about 500 μg. Alternatively, a single monthly dosage may contain both Vitamin D and 25-OH D3 each in an amount from 30 μg to about 1500 μg, preferably about 75 μg to about 500 μg. A kit may be comprised of one, two, three, four, five, six, seven, eight, nine, ten, eleven, or twelve weekly or monthly dosages.

Dosage ratios of Vitamin D to 25-OH D3 should range between 50:1 to about 1:50, more preferably from about 25:1 to about 1:25, and even more preferably from about 6:1 to about 1:6. It has been found that a dosage ratio of approximately 6:1 Vitamin D3 to 25-OH D is particularly beneficial in increasing plasma 25-OH D levels quickly (i.e. within a few hours) and maintaining elevated plateau levels.

The following non-limiting Examples are presented to better illustrate the invention.

### Example 1

Analyses were performed using baseline data of 127 participants of the ProMuscle study, which was originally designed to study the effect of protein supplementation, in combination with or without progressive exercise training on muscle function and muscle mass. Eligibility was defined as being 65 years or older and being frail or pre-frail. Frailty was defined according to the criteria from Fried et al. as having unintentional weight loss, weakness, self-reported exhaustion, slow walking speed, and/or low physical activity. A participant was classified as pre-frail when one or two of the aforementioned criteria were met, while frail was classified when three or more criteria were present. Participants were excluded if they had fasting plasma glucose (FPG) levels=7.0 mmol/L, cancer, Chronic Obstructive Pulmonary Disease, renal failure or when they participated in any structured exercise training program in the past two years. The Wageningen University Medical Ethical Committee approved the study and all participants gave their written informed consent.

### Mental Well-being

Overall cognitive functioning was assessed using the Mini Mental State Examination (MMSE). The variable for analyses was defined as the maximum MMSE score minus the MMSE score of the participant, reflecting the number of erroneous answers. Domain-specific cognitive performance was measured using an extensive cognitive test battery, performed by well-trained research assistants and according to a strict protocol. The cognitive test battery included the Word Learning Test (WLT) direct recall, decayed recall and recognition to measure episodic memory; Wechsler digit span forward and backward test to determine attention and working memory; Trail Making Test-A (TMT-A and Trail Making Test-B (TMT-B) to assess information processing speed and concept shifting interference; Stroop Color-Word Test to determine selective attention and susceptibility to behavioral interference; and the Verbal fluency test and Reaction Time Task to measure executive functioning. Apart from the Reaction Time Task, all aforementioned tests were always performed in the afternoon. Reaction time (RT) was assessed during the morning, in fasted state, by the computerized finger pre-cueing task.

### Biochemical Analyses

Blood samples were collected at baseline while participants were in a fasting state. EDTA containing tubes were centrifuged at 1000×g at 4° C. for 10 min and serum tubes were centrifuged 90 min after the blood collection at 1000×g at 18° C. for 30 min. Aliquots of plasma and serum were frozen in liquid nitrogen and stored at −80° C. until further analysis. Plasma glucose concentrations were analyzed with a COBAS FARA analyzer (Uni Kit III; Roche, Basel, Switzerland). Insulin was analyzed by radioimmunoassay (Insulin RIA Kit; LINCO Research Inc, St Charles, Mo.). Serum 25-OH D3 was measured by isotope dilution-online solid phase extraction liquid chromatography-tandem mass spectrometry (ID-XLC-MS/MS)—which was performed at the Endocrine Laboratory of the VU University Medical Center. Serum 25-OH D3 was released from its binding protein(s) and a deuterated internal standard (IS: 25-OH D33-d6) was added. Samples were extracted and analysed by XLC-MS/MS (a Symbiosis online SPE system (Spark Holland, Emmen, the Netherlands) coupled to aQuattro Premier XE tandem mass spectrometer (Waters Corp., Milford, Mass.)). Method characteristics: LOQ 4.0 nmol/L; intra-assay CV<6% and inter-assay CV<8% for 3 concentrations between 25 and 180 nmol/L.

### Dietary Intake

Dietary intake data were obtained by 3-day food records. Trained dieticians gave oral and written instructions about recording the type of foods and estimation of portion sizes in household measures. At a second visit, dieticians checked the food records for completeness, obtained additional information about unclear items or amounts, and used examples household measures to improve the estimation of portion sizes. The days of recording were randomly assigned so that all days of the week, including weekend days, were equally represented. Dietary intake data were coded (type of food, time of intake, and amount) and energy and macronutrient intakes were calculated using a nutrient calculation program (BAS nutrition software, 2004, Arnhem, the Netherlands) and the Dutch food composition database.

### Covariates

Height was measured at baseline with a wall-mounted stadiometer to the nearest 0.1 cm. Weight was measured in a fasted state to the nearest 0.1 kg with a calibrated digital scale (ED-6-T; Berkel, Rotterdam, The Netherlands). Subsequently, Body Mass Index (BMI) was calculated as weight/height². Information on education level (primary, secondary or higher education), smoking status (non-smoker or current smoker), medical history and presence of chronic disease (including kidney disease, liver disease, cardiovascular disease, muscle disease, hip- or knee replacement) was collected using questionnaires. Blood pressure was measured in the morning after 10 minutes of rest in supine position with an Omron HEM-907 (Lake Forest, Ill., USA) device. Habitual physical activity was quantified using a triaxial accelerometer (ActiGraph GTX3, 2009, Pensacola, Fla.) worn on the hip for 1 week. Change of acceleration per second and epochs of 60 seconds were used. After 7 days, data were uploaded for analysis and analysed using the MAH/UFFE analyser, version 1.9.0.3 (MRC 149 Epidemiology Unit, Cambridge, UK). Data files that did not meet 10 hours of monitoring per day on at least 5 days as well as files that included periods of >100 min without activity were excluded from the analysis.

### Statistical Analyses

Characteristics of the study population are reported using the mean with standard deviation (SD), or as percentages. Medians with interquartile range were used to report skewed variables. Chi-squared tests for categorical variables and 1-way analysis of variance for continuous variables were performed to compare baseline characteristics over tertiles of 25-OH D3. Multivariable regression analyses were performed to study the associations of 25-OH D3 with domain-specific cognitive functioning. To compare the results of the individual cognitive tests and to limit the number of dependent variables, crude test scores were clustered into compound Z-scores for four neuropsychological domains: Episodic memory, Attention and working memory, Information processing speed, and Executive functioning. In formula form:

Episodic memory=(WLT total immediate recall+WLT decayed recall+WLT recognition)/3;

attention and working memory=(Digit Span forward+Digit Span backward)/2;

information processing speed=(Average Stroop card 1 and 2+-TMT-A+-RT uncued)/3;

Executive Functioning=(Stroop ratio+Verbal Fluency+-TMT-ratio+-RT finger-cued+-RT hand-cued+-RT neither-cued)/6.

WLT decayed recall was calculated as the number of words recalled after approximately 15 minutes following the fifth session of the WLT minus the number of words recalled during the fifth session of the WLT.

Stroop ratio was calculated by: Stroop card 3/((Stroop card 1+Stroop card 2)/2).

Accuracy and speed-accuracy trade-off (SATO) were calculated to take into account errors made during the Stroop Test. Accuracy=(maximum right answers−amount of errors/maximum right answers). SATO=accuracy/time needed to complete the task. T

MT-ratio was calculated by TMT-B/TMT-A.

As MMSE-score followed a Poisson distribution, Rate Ratios (RRs) for 25-OH D3 and global cognitive performance were calculated using multivariable Poisson regression with the number of erroneous answers as outcome for global cognitive functioning. This RR corresponds to the probability of developing depression or global cognitive dysfunction in participants with either intermediate or highest 25-OH D3 levels in this population compared to participants with the lowest 25-OH D levels.

Participants were categorized according to tertiles of 25-OH D3, using the lowest tertile as the reference category. All analyses were adjusted for age, sex (model 1), BMI, education, smoking, alcohol consumption, habitual physical activity and season of blood sampling (model 2). Spearman and Pearson correlation analyses were performed to obtain correlations coefficients between vitamin D intake, 25-OH D levels, and domain specific cognitive functioning. All analyses were performed using the statistical package SAS, version 9.1 (SAS Institute Inc., Cary, N.C., USA).

### Results

Participants of the ProMuscle Study were on average 79 years old and 25-OH D levels decreased with age (P=0.03) see TABLE 1, below. The mean serum 25-OH-D level was 54 nmol/L. However, 17% of the population had 25-OH D3 levels below 30 nmol/L and 53% below 50 nmol/L. Only 23% of the participants showed 25-OH D3 levels 75 nmol/L. Mean vitamin D intake was 4.59 μg/d, with 25 percentile of 2.48 μg/d and 75 percentile of 5.78 μg/d. Calcium intake increased across 25-OH D tertiles (P=0.04).

TABLE I
Characteristics of 127 Dutch Frail Elderly per tertile of serum 25(OH)D

| | T1 | | T2 | | T3 | | |
|---|---|---|---|---|---|---|---|
| | 13-38 nmol/L | | 38-65 nmol/L | | 65-163 nmol/L | | P-value |
| N | 40 | | 43 | | 44 | | |
| Men, n (%) | 16 | (40) | 18 | (42) | 16 | (36) | 0.29 |
| Age | 81.4 ± 7.8 | | 78.7 ± 7.8 | | 77.0 ± 7.3 | | 0.03 |
| Body Mass Index | 27.8 ± 4.4 | | 28.0 ± 3.8 | | 26.8 ± 4.8 | | 0.42 |
| Fasting plasma glucose | 5.25 ± 0.50 | | 5.28 ± 0.44 | | 5.24 ± 0.48 | | 0.90 |
| (mmol/L) | | | | | | | |
| Fasting plasma insulin | 17.35 ± 6.18 | | 20.96 ± 7.46 | | 16.98 ± 6.67 | | 0.01 |
| (uU/ml) | | | | | | | |
| Homa-IR | 4.1 ± 1.8 | | 5.0 ± 2.1 | | 4.1 ± 1.9 | | 0.04 |
| Chronic disease present, | 22 | (55) | 18 | (42) | 26 | (59) | 0.25 |
| n (%) | | | | | | | |
| Systolic Blood Pressure | 152 ± 22 | | 144 ± 22 | | 144 ± 23 | | 0.21 |
| (mmHg) | | | | | | | |
| Diastolic Blood Pressure | 75 ± 9 | | 75 ± 9 | | 74 ± 9 | | 0.76 |
| (mmHg) | | | | | | | |
| Serum creatinine | 76.6 ± 15.6 | | 74.5 ± 13.1 | | 70.9 ± 15.6 | | 0.21 |
| (mmol/L) | | | | | | | |
| Smoking status, n (%) | | | | | | | 0.07 |
| Non-smoker | 35 | (88) | 43 | (100) | 40 | (91) | |
| Smoker | 5 | (12) | 0 | (0) | 4 | (9) | |
| Physical activity | 91 ± 62 | | 136 ± 75 | | 184 ± 112 | | 0.0001 |
| accelerometer | | | | | | | |
| Educational level, n (%) | | | | | | | 0.26 |
| Primary education | 1 | (3) | 3 | (7) | 3 | (7) | |
| Secondary education | 28 | (70) | 20 | (46.5) | 27 | (61) | |
| Higher education | 11 | (28) | 20 | (46.5) | 14 | (32) | |
| Vitamin D intake | 3.2 | (1.7-5.7) | 3.9 | (2.5-5.0) | 4.4 | (3.0-7.3) | 0.21 |
| (μg/day) | | | | | | | |
| Calcium intake (mg/day) | 921 ± 343 | | 1029 ± 322 | | 1135 ± 476 | | 0.04 |
| Alcohol intake (g/day) | 4.5 | (0-12.2) | 6.3 | (0.1-19.7) | 7.0 | (0-20.2) | 0.35 |

| | | | | |
|---|---|---|---|---|
| MMSE | 27.5 ± 2.3 | 27.7 ± 2.2 | 27.8 ± 2.2 | 0.81 |
| Attention and working | −0.16 ± 0.80 | 0.00 ± 0.86 | 0.14 ± 0.99 | 0.31 |
| memory | | | | |
| Executive functioning | −0.15 ± 0.83 | −0.07 ± 0.74 | 0.25 ± 0.62 | 0.04 |
| Information processing | −0.22 ± 0.77 | −0.03 ± 0.87 | 0.24 ± 0.74 | 0.05 |
| speed | | | | |
| Episodic memory | −0.04 ± 0.76 | −0.11 ± 0.58 | 0.10 ± 0.64 | 0.36 |
| CES-D | 5.55 ± 5.33 | 7.42 ± 4.79 | 7.77 ± 6.77 | 0.17 |

Note:
values are expressed as a mean ± SD, median with Q1-Q3 or n (%).
Chi-squared tests for categorical variables and 1-way analysis of variance for continuous variables were performed to compare baseline characteristics over tertiles of 25(OH)D.
Chronic disease: defined as kidney disease, liver disease, cardiovascular disease, muscle disease, hip- or knee replacement.
Missing values: physical activity (21), executive function (9), information processing speed (8), fasting plasma insulin, fasting plasma glucose and Homa-IR (3) and blood pressure and creatinine (1).

Vitamin D intake was not significantly correlated with serum 25-OH D3, r=0.09 (P=0.30).

### Vitamin D and Cognitive Performance

Compared to participants in the lowest 25-OH D3 tertile, persons in the intermediate and top tertile scored higher on the MMSE, this finding was, however, not significant (see TABLE 2, below). Associations between 25-OH D3 and various cognitive domains are displayed in TABLE 3, below.

### Vitamin D and Cognitive Performance

Compared to participants in the lowest 25-OH D3 tertile, persons in the intermediate and top tertile scored higher on the MMSE, this finding was, however, not significant (see TABLE 2, below). Associations between 25-OH D3 and various cognitive domains are displayed in TABLE 3, below.

TABLE 2
Associations between 25(OH)D and global cognitive performance
(MMSE) of 127 elderly participating in the ProMuscle Study

| | Low | Moderate | High | |
|---|---|---|---|---|
| | 0-34 | 34-52 | 52-125 | P for trend |
| Crude model | 1.0 | 0.93 (0.62-1.38) | 0.87 (0.58-1.31) | 0.79 |
| Model 1^{a} | 1.0 | 1.00 (0.68-1.47) | 0.98 (0.65-1.47) | 0.96 |
| Model 2^{b} | 1.0 | 0.83 (0.55-1.24) | 0.78 (0.51-1.20) | 0.77 |

^{a}Adjusted for age and sex.
^{b}Adjusted for age, sex, BMI, education (categorical), smoking (categorical), physical activity accelerometer, alcohol intake (categorical) and season
Crude model and model 1: n = 127. Model 2: n = 106.

TABLE III
Associations of 25(OH)D and vitamin D intake with domain-specific cognitive
performance in a frail elderly population, β ± SE

| Total | | ‘Low’ FPG | ‘High’ FPG |
|---|---|---|---|
| 25(OH)D | Vitamin D intake^{c} | 25(OH)D | 25(OH)D |

Attention and working memory

| | | | | |
|---|---|---|---|---|
| Crude | 0.006 ± 0.003 | −0.007 ± 0.03 | 0.004 ± 0.003 | 0.005 ± 0.005 |
| | (P = 0.06) | (P = 0.78) | (P = 0.19) | (P = 0.17) |
| Model 1 | 0.005 ± 0.003 | −0.013 ± 0.03 | 0.004 ± 0.003 | 0.007 ± 0.006 |
| | (P = 0.08) | (P = 0.62) | (P = 0.26) | (P = 0.23) |
| Model 2 | 0.006 ± 0.004 | −0.02 ± 0.03 | 0.006 ± 0.004 | 0.008 ± 0.008 |
| | (P = 0.11) | (P = 0.46) | (P = 0.17) | (P = 0.31) |

Executive functioning

| | | | | |
|---|---|---|---|---|
| Crude | 0.007 ± 0.002 | 0.002 ± 0.02 | 0.007 ± 0.003 | 0.010 ± 0.004 |
| | (P = 0.003) | (P = 0.94) | (P = 0.04) | (P = 0.01) |
| Model 1 | 0.006 ± 0.002 | −0.01 ± 0.02 | 0.005 ± 0.003 | 0.009 ± 0.004 |
| | (P = 0.01) | (P = 0.65) | (P = 0.11) | (P = 0.03) |
| Model 2 | 0.007 ± 0.003 | −0.05 ± 0.02 | 0.006 ± 0.004 | 0.007 ± 0.005 |
| | (P = 0.01) | (P = 0.06) | (P = 0.12) | (P = 0.17) |

Information processing speed

| | | | | |
|---|---|---|---|---|
| Crude | 0.007 ± 0.003 | 0.03 ± 0.02 | 0.007 ± 0.004 | 0.01 ± 0.004 |
| | (P = 0.01) | (P = 0.24) | (P = 0.07) | (P = 0.02) |
| Model 1 | 0.006 ± 0.003 | 0.02 ± 0.02 | 0.006 ± 0.004 | 0.008 ± 0.004 |
| | (P = 0.03) | (P = 0.47) | (P = 0.14) | (P = 0.05) |
| Model 2 | 0.006 ± 0.003 | 0.01 ± 0.03 | 0.006 ± 0.004 | 0.004 ± 0.005 |
| | (P = 0.06) | (P = 0.59) | (P = 0.16) | (P = 0.42) |

Episodic memory

| | | | | |
|---|---|---|---|---|
| Crude | 0.003 ± 0.002 | 0.009 ± 0.02 | 0.003 ± 0.003 | 0.004 ± 0.004 |
| | (P = 0.14) | (P = 0.65) | (P = 0.25) | (P = 0.24) |
| Model 1 | 0.002 ± 0.002 | −0.002 ± 0.02 | 0.003 ± 0.003 | 0.002 ± 0.004 |
| | (P = 0.47) | (P = 0.93) | (P = 0.49) | (P = 0.67) |
| Model 2 | 0.002 ± 0.002 | −0.03 ± 0.02 | 0.003 ± 0.003 | 0.001 ± 0.004 |
| | (P = 0.28) | (P = 0.17) | (P = 0.30) | (P = 0.74) |

^{a}Model 1: Adjusted for age and gender
^{b}Model 2: Adjusted for model 1 and BMI, education level, alcohol, smoking, physical activity based upon 7-day accelerometer data and season
^{c}Analysis for vitamin D intake are not adjusted for seasonal influences Crude model and model 1: n = 127 (AWM, EM), n = 118 (EF) and n = 119 (IPS). Model 2: n = 106 (AWM, EM) and n = 99 (EF, IPS).

Fully adjusted models showed significantly better performance in tasks involving executive functioning per 1 nmol/L increase in 25-OH D, R 0.007 (P=0.01). The association between 25-OH D3 and information processing speed almost reached significance, R 0.006 (P=0.06). When the models were expanded to capture the impact of depression or calcium R's did not significantly change (data not shown).

Within the domain executive functioning the association of 25-OH D3 nearly reached significance with the verbal fluency test, showing on average 1 word more recalled with every 17 nmol/L increase in 25-OH D3 (P=0.09). Also a modest association was observed for 25-OH D3 with the Reaction Time Task (neither cued: β—2.86, p=0.01; finger cued: β—2.71, P=0.01; hand cued: β—2.86, P=0.01).

Serum 25-OH D3 showed a significant association with the reaction time task uncued (β—2.58, p=0.01), a task related to the domain information processing speed.

Additionally, a modest association was observed for 25-OH D3 with Digit Span (R 0.02, p=0.07), within the domain attention and working memory.

Scores on the Word Learning Tests indicated that with every 11 nmol/L increase in 25-OH D3 one word more could be memorized (P=0.008).

We did not detect any association between vitamin D intake and the various outcome measures. Moreover, correlations between vitamin D intake and 25-OH D3 appeared to be low. In this population we observed a statistical significant association between 25-OH D3 and the domain executive functioning. In this study executive function was assessed using tasks related to response inhibition, cognitive flexibility and mental shifting.

### Example 2

### The Effect of 25(OH) Vitamin D3 on Executive Function

### A Double-Blind, Randomized, Placebo-Controlled Trial in the Frail Elderly

### Primary and Secondary Objectives

The primary objective of the study is to assess the effect of 25(OH) D in comparison to Vitamin D3 on executive function in frail elderly with a low Vitamin D status. The secondary objective is to assess 25-OH D3 effect on increasing 25-OH D3 serum level in comparison to Vitamin D3.

### Study Design, Participants and Treatment

The design is a parallel group, randomized, placebo-controlled, double-blind study with three different application groups. Each group has an appropriate number of participants (e.g., 20 participants). Eligibility is defined as being 65 years or older and being frail or pre-frail. Frailty is defined as having unintentional weight loss, weakness, self-reported exhaustion, slow walking speed, and/or low physical activity (see Fried L P, et al. Frailty in older adults: evidence for a phenotype. J Gerontol A Biol Sci Med. Sci. March 2001; 56(3):M146-156.)

A participant is classified as pre-frail when one or two of the aforementioned criteria are met, while frail is classified when three or more criteria are present. The study subjects have to have a plasma 25-OH D3 levels below 50 mmol/L. The treatment conditions are either 25-OH D3, Vitamin D3 (dry Vitamin D3 100 SD/S), the combination of 25-OH D3 and Vitamin D3, or placebo. The duration of supplementation is 6 months (180 days).

### Study Procedures

Participants are randomly allocated to either the 25-OH D3 group (N=20), Vitamin D3 group (N=20), Vitamin D3+25 OH D3 group (N=20) or placebo group (N=20). Double-blind randomization is carried out by means of a label code associated with a specific participant ID number, whose meaning is known only to the supplier. Participants are allocated randomly to these numbers.

At screening (in between Day −14 to Day 0) the following tests are performed to screen for serum 25-OH D3 levels below 50 mmol/L: Serum: 25-OH D3, VD3, calcium, creatinine, albumin; urine: calcium and creatinine; blood pressure.

For baseline (Day 0) the following tests are performed: Physical examination, vital signs (blood pressure, heart rate), demographics, clinical laboratory, FFQ, serum: 25-OH D3, 24,25-OH 2D, 1,25(OH)2D3, Vitamin D3, calcium, albumin, creatinine, fasting glucose, PTH; urine: calcium, creatinine; executive function tests (see below). Baseline evaluation is followed by the randomization of the subjects. From Day 2 on, they start to take the study compounds. Daily dosages for those receiving 25-OH D3 are 5-25 μg per day or 35-175 μg per week. For Vitamin D3, dosages range from 5-25 μg per day, or 35-175 μg per week. For the combination administration, the same ranges of both actives are administered.

At visits on Day 90 and Day 180 the following tests are performed: Vital signs (blood pressure, heart rate), serum: calcium, creatinine, albumin, 25-OH D3, 24,25(OH)2D, 1,25(OH)2D3, VD3, fasting glucose; urine: calcium and creatinine; compliance; executive function tests.

In addition, at visit on Day 180 the following tests are performed: Physical examination, clinical laboratory, serum PTH, urine DPD.

Executive functioning test batteries include the following standard tests of executive functioning: Trail Making Tests A and B (to determine the ratio of TMT-B/TMT-A), Stroop Colour-Word Test, including determining the Stroop ratio, calculated by Stroop card 3/((Stroop card 1+Stroop card 2)/2) and animal fluency test. These tests are always performed at the same time of the day.

### Results

25(OH) Vitamin D3 has a significantly higher effect on the improvement of executive functioning in frail elderly in comparison to Vitamin D3 and placebo, respectively. Also, the group receiving 250H D3+Vitamin D3 is seen to have a higher sustained plasma 25-OH D3 level than groups receiving either active alone; and thus use of this combination in a weekly dose is also effective.

Furthermore, it is being shown that administration of 25-OH D3 significantly increases 25-OH D serum level in comparison to administration of only Vitamin D3.

### Example 3

### The Effect of 25(OH) Vitamin D3 on Executive Function

### A Double-Blind, Randomized, Placebo-Controlled Trial in the General Adult Population

### Study Design, Participants and Treatment

The design is a parallel group, randomized, placebo-controlled, double-blind study similar to that of Example 2, above, except that the participants are healthy adults under 65. The duration of supplementation is 6 months (180 days). Group size is chosen so that a statistically significant difference, if present, may be observed between placebo and test groups.

### Study Procedures

Participants are randomly allocated to either the 25-OH D3 group, Vitamin D3 group, (Vitamin D3+25 OH D3) group or placebo group. Double-blind randomization is carried out by means of a label code associated with a specific participant ID number, whose meaning is known only to the supplier. Participants are allocated randomly to these numbers.

For baseline (Day 0) the following tests are performed: Physical examination, vital signs (blood pressure, heart rate), demographics, clinical laboratory, FFQ, serum: 25-OH D3, 24,25(OH)2D, 1,25(OH)2D3, Vitamin D3, calcium, albumin, creatinine, fasting glucose, PTH; urine: calcium, creatinine; executive function tests (see below). Baseline evaluation is followed by the randomization of the subjects. From Day 2 on, they start to take the study compounds.

At visits on Day 90 and Day 180 the following tests are performed: Vital signs (blood pressure, heart rate), serum: calcium, creatinine, albumin, 25-OH D3, 24,25(OH)2D, 1,25(OH)2D3, VD3, fasting glucose; urine: calcium and creatinine; compliance; executive function tests.

In addition, at visit on Day 180 the following tests are performed: Physical examination, clinical laboratory, serum PTH, urine DPD.

Executive functioning test batteries include the following standard tests of executive functioning: Trail Making Tests A and B (to determine the ratio of TMT-B/TMT-A), Stroop Colour-Word Test, including determining the Stroop ratio, calculated by Stroop card 3/((Stroop card 1+Stroop card 2)/2) and animal fluency test. These tests are always performed at the same time of the day.

### Results

Positive results are seen in comparison to placebo. The group receiving a weekly dose of the combination is also significantly different from placebo.

Furthermore, it is being shown that administration of 25-OH D3 significantly increases 25-OH D serum level in comparison to administration of only Vitamin D3.

### Example 4

### Daily Dosage Kit

A package suitable for over the counter consumer marketing is manufactured. It comprises:

a) a cardboard box containing two blister packs of 25-OH D3 tablets each, for a total of 30 tablets. Each tablet contains between 5-25 μg of 25-OH D3, which are to be taken once per day.

b) a package insert which informs the consumer that 25-OH D3 serum blood levels are associated with the maintenance of cognitive functioning in healthy adults.

The packaging may be modified to include a desired number (such as 3, 4, 5, or 6) weekly dosage forms or one or more monthly dosage forms. The information provided on the package insert may be printed on the box. A website is set up which provides examples of how 25-OH D3 helps maintain cognitive function for healthy people in a variety of occupations where executive functioning is required.

What is claimed is:

1. A method of marketing 25-hydroxyvitamin D3 (“25-OH D3”) to maintain or enhance executive functioning in healthy individuals or lessen the decrease of executive functioning in healthy individuals comprising:
a) providing an effective amount of 25-OH D3 to a person desirous of maintaining or enhancing executive functioning, or lessening the risk of a decline in executive functioning; and
b) informing the healthy individuals of the ability of 25-OH D3 maintain or enhance executive functioning in healthy individuals or lessen the risk of a decline in executive functioning.

2. A method according to claim 1 wherein the 25-OH D3 is provided in a kit.

3. A method according to claim 1 wherein the 25-OH D3 is in an oral formulation.

4. A method according to claim 1 comprising further providing vitamin D3.

5. A method according to claim 1 wherein an additional active ingredient is also provided.

6. A method according to claim 1 wherein multiple daily dosages of 25-OH D3 are provided.

7. A method according to claim 1 wherein at least one weekly dose of 25-OH D3 is provided.

8. A method according to claim 1 wherein at least one monthly dose of 25-OH D3 is provided.
